(19) United States
(12) Patent Application Publication
(10) Pub. No.: US 2013/0253377 A1
(43) Pub. Date: Sep. 26, 2013

(54) APPARATUS FOR DETECTING A STRENGTH OF LIPS-CLOSING AND METHOD THEREOF

(71) Applicant: NATIONAL TAIWAN UNIVERSITY OF SCIENCE AND TECHNOL, Taipei (TW)

(72) Inventors: YUAN-HSIANG LIN, Taipei (TW); SHIN-YU GAU, Taipei (TW); WEI-CHUN HSU, Taipei (TW)

(73) Assignee: National Taiwan University of Science and Technology, Taipei (TW)

(21) Appl. No.: 13/675,067

(22) Filed: Nov. 13, 2012

(30) Foreign Application Priority Data
Mar. 21, 2012 (TW) 101109670

(51) Int. Cl. A61B 5/22; A61B 5/00; A61B 5/11

(52) U.S. Cl. CPC A61B 5/224; A61B 5/11; A61B 5/742; A61B 5/486; A61B 5/7225; A61B 5/7278
USPC 600/590

(57) ABSTRACT

An apparatus for detecting a strength of lips-closing is applied for training and evaluation during treatment of temporomandibular disorders (TMD), according to the strength of the lips-closing of the user, the clinical staff and the patients are able to evaluate the severity of lips-closing impairment. It can also be used as training tool with which the effect can be monitored before, during, and after rehabilitation courses. The apparatus includes an air bag, a pressure sensing unit and a micro-controller module. The user puts the air bag, which is able to be pumped air in or out, between the lips. The pressure sensing unit is connected to the air bag for detecting a pressure formed by the lips of the user forcing the air bag, to generate a signal. The micro-controller module is electrically connected to the pressure sensing unit for converting the signal into the strength of lips-closing.

### BACKGROUND OF THE INVENTION

(1) Field of the Invention

The invention relates to a medical detecting apparatus, and especially relates to an apparatus for detecting a strength of lips-closing and a method thereof for training and evaluation during treatment of temporomandibular disorders (TMD).

(2) Description of the Prior Art

The temporomandibular disorders refer to the disease of the muscles of the temporomandibular joint (TMJ) or jaw function including mouth opening and closing. For the treatment of TMD, mainly by physical therapy to relieve pain and restore normal joint function, and its treatments include the following manner: self-practice for muscle relaxation, the use of cold spray to inhibit pain, compression with cold or hot packs and other ways to carry out the treatment. Lips dysraphism is one of the key treatment issues in the rehabilitation process. Therefore, during treatment of TMD, if the patients can immediately understand their own strength of lips-closing of lips closed, it is helpful to assist recording their training. On one hand, it can help clinical staff to understand the recovery of their patients; on the other hand, it can provide instantaneous feedback of the training process in patients.

However, there is no tool used to measure the strength of lips-closing. Modern medical equipment, only one kind can be measured teeth bite together to the device, it has to assist the staff to understand the function of patients with occlusion. This device is attached by a pressure sensing tablets as a sensor to measure the teeth bite force of the patients. The pressure sensing tablets permit a direct measurement on the strength of the force contact area, because of the different positions of the load to slight changes. In addition, because the value of the strength of the teeth, namely bite strength, relative to lip force strength is far greater, more accurate tool for the strength of lips-closing measurements is required.

Although the conventional pressure sensing tablets can easily obtain the teeth bite strength, the pressure sensing tablet is often produce little error due to changes in the location, resulting in the difficulties to accurately measure a strength of lips-closing. Therefore, to solve the problem, the technique desperately wants to provide the medical testing devices which can precisely measure the strength of lips-closing.

### SUMMARY OF THE INVENTION

The object of the invention is to provide an apparatus for detecting a strength of lips-closing, applied for training and evaluation during treatment of TMD. According to the strength of the lips-closing of the user, the clinical staff and the patients are able to evaluate the severity of lips-closing impairment.

The other objects and advantages can be disclosed from the technical features to get a better understanding in this invention.

In order to achieve one of the above or in part or all of the objects or other objects, the apparatus for detecting a strength of lips-closing in one embodiment comprises an air bag, a pressure sensing unit and a micro-controller module. The air bag, which can be inflated or deflated by controlling a pump, is placed between the lips of a user. The pressure sensing unit is connected to the air bag to detect the lips of the user for sensing a pressure produced in the air bag, and so as to produce a signal. The micro-controller module is electrically connected to the pressure sensing unit, to convert the signal into strength of lips-closing.

In an embodiment, the apparatus further comprises a pump and a deflating valve, which are electrically connected to the micro-controller module. The micro-controller module controls the pump to inflate the air bag, or controls the deflating valve to deflate the air bag.

In another embodiment, the apparatus further comprises an amplification unit, electrically connected between the pressure sensing unit and the micro-controller module, and to amplify the signal generated by the pressure sensing unit.

In another embodiment, the apparatus further comprises a filter unit, electrically connected between the pressure sensing unit and the micro-controller module, and to filter the signal generated by the pressure sensing unit.

In another embodiment, the apparatus further comprises a user interface electrically connected the micro-controller module. The user can view an operation window and control the micro-controller module through the user interface, thereby control the air bag inflated or deflated for adjusting the pressure therein, in order to achieve a predetermined pressure value.

In another embodiment, the apparatus further comprises a record unit. The record unit records a lips-closing time point and a lips-closing duration when the user forcing the air bag, and also records an opening-closing frequency as the lips of the user opening and closing on the air bag. Moreover, the record unit records the strength of lips-closing processed by the micro-controller module, thereby obtain a current strength of lips-closing and a maximum strength of lips-closing. Then, the record unit stores the closing duration, the opening-closing frequency, the current strength of lips-closing and the maximum strength of lips-closing.

In order to achieve one of the above or in part or all of the objects or other objects, a method for detecting the strength of lips-closing in one embodiment comprises steps of: providing the above apparatus for detecting the strength of lips-closing; setting the predetermined pressure value of the air bag; controlling the inflating or deflating procedure of the air bag according to the predetermined pressure value; placing the air bag within the lips of the user when the pressure in the air bag reaches the predetermined pressure value; sensing the pressure exerted on the air bag by the lips of the user to generate a signal; and, converting the signal into the strength of lips-closing.

In an embodiment, the method further comprises a step of inflating or deflating the air bag according to the predetermined pressure value.

In another embodiment, the method further comprises steps of: amplifying the signal and filtering the amplified signal.

In another embodiment, the method further comprises steps of: recording a closing duration and an opening-closing frequency of the lips; recording the strength of lips-closing processed by the micro-controller module, thereby obtain the current strength of lips-closing and the maximum strength of lips-closing; and storing the closing duration, the opening-closing frequency, the current strength of lips-closing and the maximum strength of lips-closing in the database.

In another embodiment, the user interface has a real-time visual feedback mechanism, and the method further comprises steps of: forming a real-time force curve according to the strength of lips-closing; and providing a real-time visual feedback mechanism according to the real-time force curve. The real-time visual feedback mechanism comprises steps of: displaying two real-time patterns on the user interface to designate an upper bound and a lower bound of the real-time force curve; and displaying the relative position of the real-time force curve, the upper bound and the lower bound on the user interface as the lips of the user exerting the pressure on the air bag. Therefore, the user can set an upper bound and a lower bound of the real-time three curve through the user interface; and designate the upper bound and the lower bound respectively via the two real-time patterns. When the lips of the user exert the pressure on the air bag, the user can control the strength of lips-closing to maintain the real-time force curve between the upper bound and the lower bound.

### BRIEF DESCRIPTION OF THE DRAWINGS

FIG. 1 is a schematic view showing a block diagram of an apparatus for detecting a strength of lips-closing in accordance with an embodiment of the present invention.

FIG. 2 is a detailed block diagram of an apparatus for detecting a strength of lips-closing in accordance with an embodiment of the present invention.

FIGS. 3 and 3A to 3C are flow charts of a method of detecting a strength of lips-closing in accordance with an embodiment of the present invention.

FIG. 4 is a schematic view showing an user interface in accordance with an embodiment of the present invention.

### DESCRIPTION OF THE PREFERRED EMBODIMENTS

In the following detailed description of the preferred embodiments, reference is made to the accompanying drawings which form a part hereof, and in which is shown by way of illustration specific embodiments in which the invention may be practiced. In this regard, directional terminology, such as “top,” “bottom,” “front,” “back,” etc., is used with reference to the orientation of the Figure(s) being described. The components of the present invention can be positioned in a number of different orientations. As such, the directional terminology is used for purposes of illustration and is in no way limiting. On the other hand, the drawings are only schematic and the sizes of components may be exaggerated for clarity. It is to be understood that other embodiments may be utilized and structural changes may be made without departing from the scope of the present invention. Also, it is to be understood that the phraseology and terminology used herein are for the purpose of description and should not be regarded as limiting. The use of “including,” “comprising,” or “having” and variations thereof herein is meant to encompass the items listed thereafter and equivalents thereof as well as additional items. Unless limited otherwise, the terms “connected,” “coupled,” and “mounted” and variations thereof herein are used broadly and encompass direct and indirect connections, couplings, and mountings. Similarly, the terms “facing,” “faces” and variations thereof herein are used broadly and encompass direct and indirect facing, and “adjacent to” and variations thereof herein are used broadly and encompass directly and indirectly “adjacent to”. Therefore, the description of “A” component facing “B” component herein may contain the situations that “A” component facing “B” component directly or one or more additional components is between “A” component and “B” component. Also, the description of “A” component “adjacent to” “B” component herein may contain the situations that “A” component is directly “adjacent to” “B” component or one or more additional components is between “A” component and “B” component, Accordingly, the drawings and descriptions will be regarded as illustrative in nature and not as restrictive.

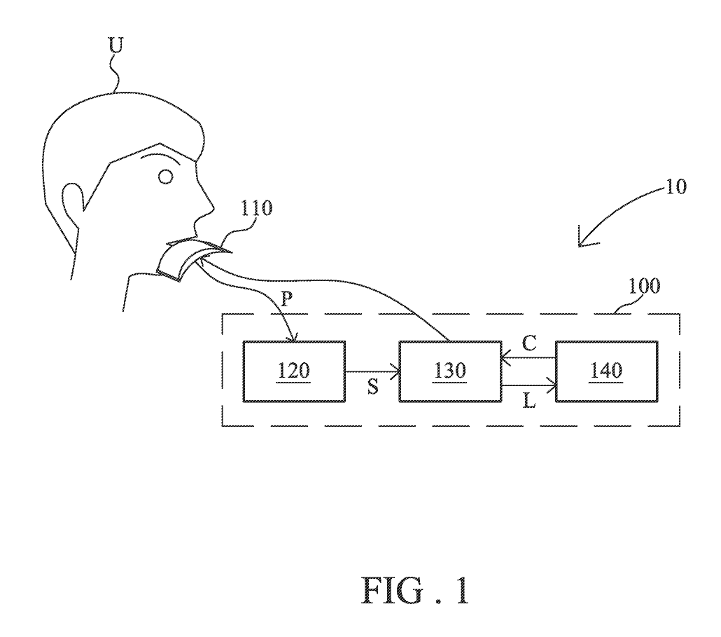

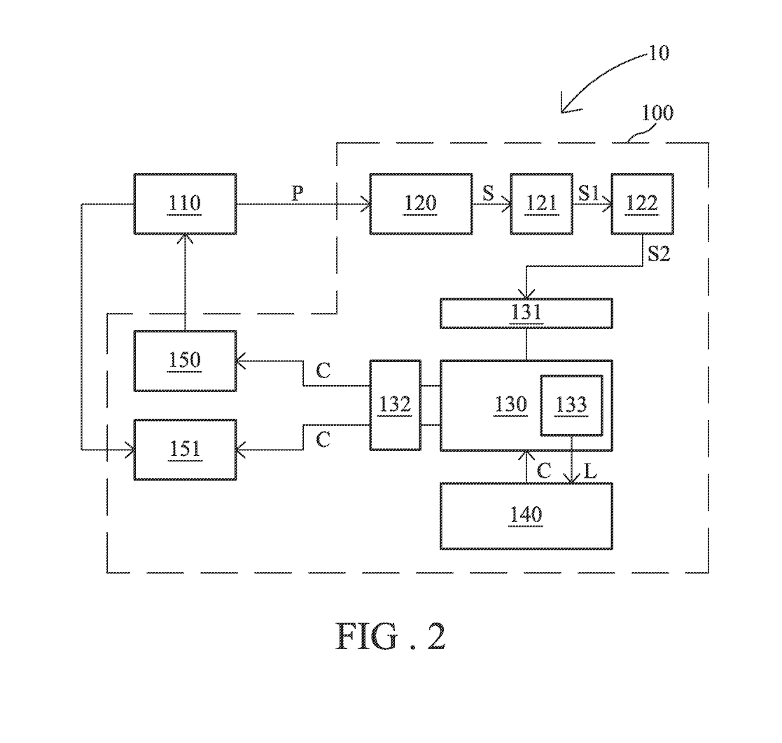

FIG. 1 is a schematic view showing a block diagram of an apparatus for detecting a strength of lips-closing L in accordance with an embodiment of the present invention. The apparatus 10 for detecting the strength of lips-closing L is applied for training and evaluation during treatment of temporomandibular, and accurately records the strength of the lips-closing L of an user U. The apparatus 10 comprises an air bag 110 and a main body 100. The main body 100 comprises a pressure sensing unit 120, a micro-controller module 130 and an user interface 140.

The air bag 110 is adapted to be placed between the lips of the user U, and can be inflated and deflated at different pressure level. The pressure sensing unit 120 is connected to the air bag 110 to detect a pressure P exerted on the air bag by closing the lips of the user U, thereby produce a signal S. The micro-controller module 130, such as a system on chip (Soc), is electrically connected to the pressure sensing unit 120, in order to convert the signal S into the strength of lips-closing L. The user interface 140 is electrically connected to the micro-controller module 130, for the user U to view. The medical staff input a controlling signal C by the user interface 140 to operate the micro-controller module 130. According to the controlling signal C, the micro-controller module 130 controls the air bag 110 inflated and deflated, causing the air bag 110 producing a reference gas pressure therein.

When the reference gas pressure in the air bag 110 reaches a predetermined pressure value, the user U can place the air bag 110 between the lips and close the lips to force the pressure P on the air bag 110. The pressure sensing unit 120 may sense a variation of the pressure P so as to generate the signal S. The signal S can be processed by the micro-controller module 130 processing an algorithm, so as to produce the strength of lips-closing L. Thereby, the medical staff is informed that the strength of lips-closing L measured by the user U closing his/her lips, and further analyzes the treatment status of the TMD.

FIG. 2 is a detailed block diagram of an apparatus for detecting the strength of lips-closing L. In an embodiment, the apparatus 10 for detecting the strength of lips-closing L further comprises an amplification unit 121, a filter unit 122, a pump 150 and a deflating valve 151. The amplification unit 121 and the filter unit 122 are electrically connected between the pressure sensing unit 120 and the micro-controller module 130. The amplification unit 121 amplifies the signal S generated by the pressure sensing unit 120. The filter unit 122 is used to filter the amplified signal S1 to form a filtered signal S2. The filtered signal S2 is sent to the micro-controller module 130 through an analog to digital converter 131. By the user interface 140, the medical staff can input the controlling signal C to the micro-controller module 130. The micro-controller module 130 sends the controlling signal C to the pump 150 and deflating valve 151 through a universal input/output terminal 132. The pump 150 and the deflating valve 151 are electrically connected to the micro-controller module 130 and connected to the air bag 110, causing the pump 150 and the deflating valve 151 making the air bag 110 inflated or deflated based on the controlling signal C in order to achieve the predetermined pressure value.

In this embodiment, the microcontroller module 130 comprises a record unit 133. According to the strength of lips-closing L processed by the micro-controller module 130, the record unit 133 records a closing duration as the lips of the user U forcing the pressure on the air bag, and records an opening-closing frequency of the lips on the air bag 110, and record all the strength of lips-closing L processed by the micro-controller module 130. Thereby, a current strength of lips-closing and a maximum strength of lips-closing are listed, and the closing duration, the opening-closing frequency, the current strength of lips-closing and the maximum strength of lips-closing are stored in a database. In this embodiment, all the strength of lips-closing L processed by the microcontroller module 130 are not limited to be provided for the above parameter calculation of the closing duration, the opening-closing frequency, the current strength of lips-closing and the maximum strength of lips-closing. According to the needs of the medical staff with a variety of algorithms to obtain the other parameters of medical staff required.

Note that the record unit 133 is not limited to be disposed in the microcontroller module 130. In another embodiment, the record unit 133 and a database may be disposed in an electronic device such as a personal computer, and be controlled by the user interface 140. In other words, the user U may operate the user interface 140 to record the strength of lips-closing L, the closing duration and the opening-closing frequency of the lips in the record unit 133 of the personal computer, thereby obtain a current strength of lips-closing and a maximum strength of lips-closing, to store the closing duration, the opening-closing frequency, the current strength of lips-closing and the maximum strength of lips-closing in the database.

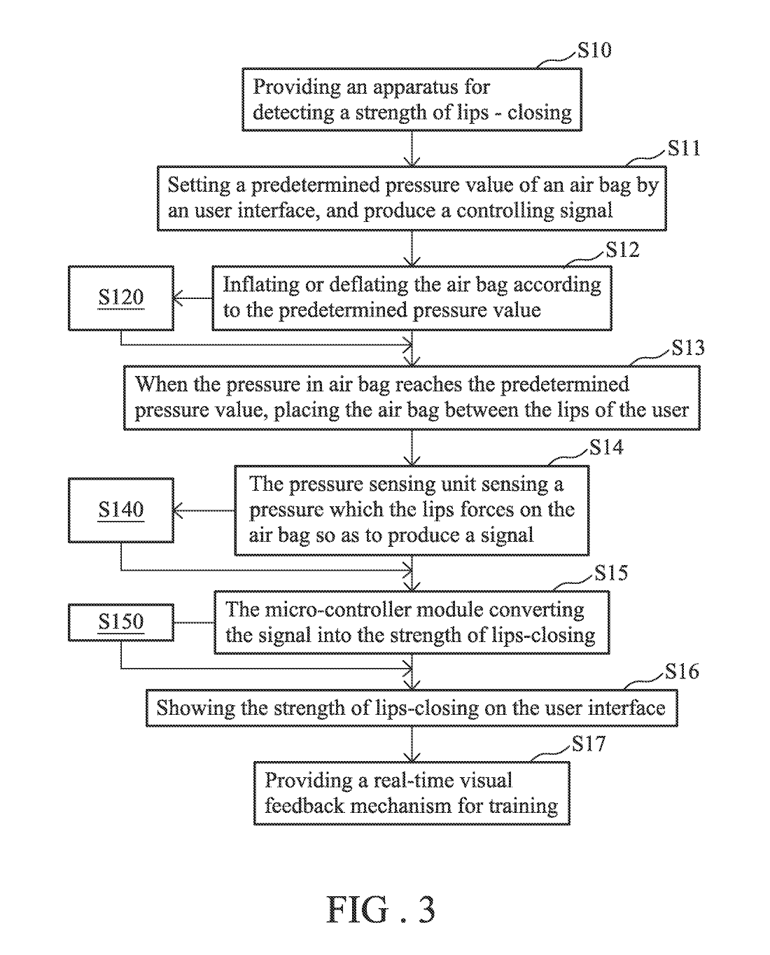

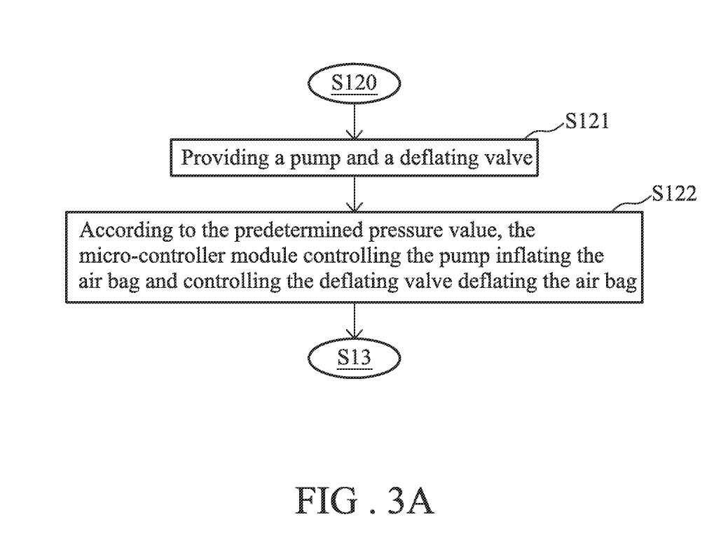

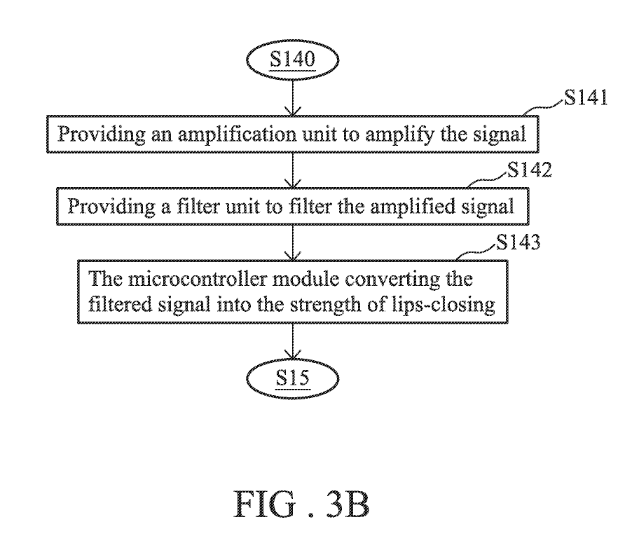

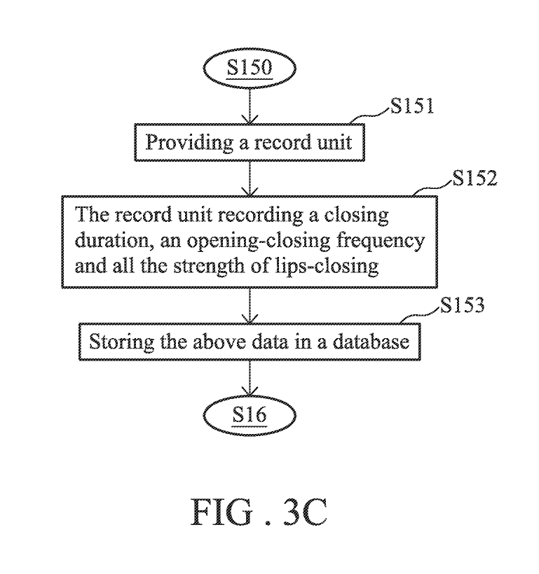

FIG. 3 is a flow chart of a method of detecting the strength of lips-closing L in accordance with an embodiment of the present invention. The method comprises the steps listed as follows:

step S10: providing the above-mentioned apparatus 10 for detecting the strength of lips-closing L;

step S11: setting the predetermined pressure value of the air bag 110 by the user interface 140, and producing the controlling signal C;

step S12: controlling the air bag 110 inflated and deflated by the controlling signal C according to the predetermined pressure value;

step S13: placing the air bag 110 between the lips of the user U when the pressure in the air bag 110 reaches the predetermined pressure value;

step S14: sensing the user U forcing the pressure on the air bag 110 by the pressure sensing unit 120 so as to generate a signal;

step S15: converting the signal into the strength of lips-closing L by the micro-controller module 130 through the algorithm process;

step S16: displaying the strength of lips-closing L in the user interface 140 for informing the medical staff that the strength of lips-closing L measured by the user U closing his/her lips, and further analyzes the treatment status of the TMD. In addition, the user interface 140 can also display a real-time force curve C₁ according to the lips strength, and thus available for real-time feedback during a patient training process; and

step S17: finally, providing a real-time visual feedback mechanism for patient training. For example, providing some real-time patterns with the real-time force curve, users U are able to better control their strength during lips-closing. According to the real-time patterns shown on the user interface 140 on a monitor, which correspond to individuals' level of the lips-closing strength, it is helpful for the users U to manipulate the fine motor control of lips motion during therapeutic training sessions.

In an embodiment, the step S12 of the method of apparatus for detecting the strength of lips-closing L, includes a flowchart shown as the step S120 in FIG. 3A, which comprises:

step S121: providing a pump 150 and a deflating valve 151; and

step S122: according to the predetermined pressure value, controlling the pump 150 inflating or/and controlling the deflating valve 151 deflating the air bag 110 by the micro-controller module 130.

In an embodiment, the step S14 of the method of apparatus for detecting the strength of lips-closing L, includes a flowchart shown as the step S140 in FIG. 3B, which comprises:

step S141: providing an amplification unit 121 for amplifying the signal S generated by the pressure sensing unit 120.

step S142: providing a filter unit 122 for filtering the signal S1 amplified through the amplification unit 121; and

step S143: converting the filtered signal S2 into the strength of lips-closing L through the microcontroller module 130.

In an embodiment, the step S15 of the method of apparatus for detecting the strength of lips-closing L, includes a flowchart shown as the step S150 in FIG. 3C, which comprises:

step S151: providing a record unit 133;

step S152: when the lips of the user U force the air bag, recording a closing duration and an opening-closing frequency of the lips based on the strength of lips-closing L by means of the record unit 133, and recording all the strength of lips-closing L processed by the micro-controller module 130, thereby obtain a current strength of lips-closing and a maximum strength of lips-closing; and

step S153: storing the closing duration, the opening-closing frequency, the current strength of lips-closing and the maximum strength of lips-closing in a database by the record unit 133.

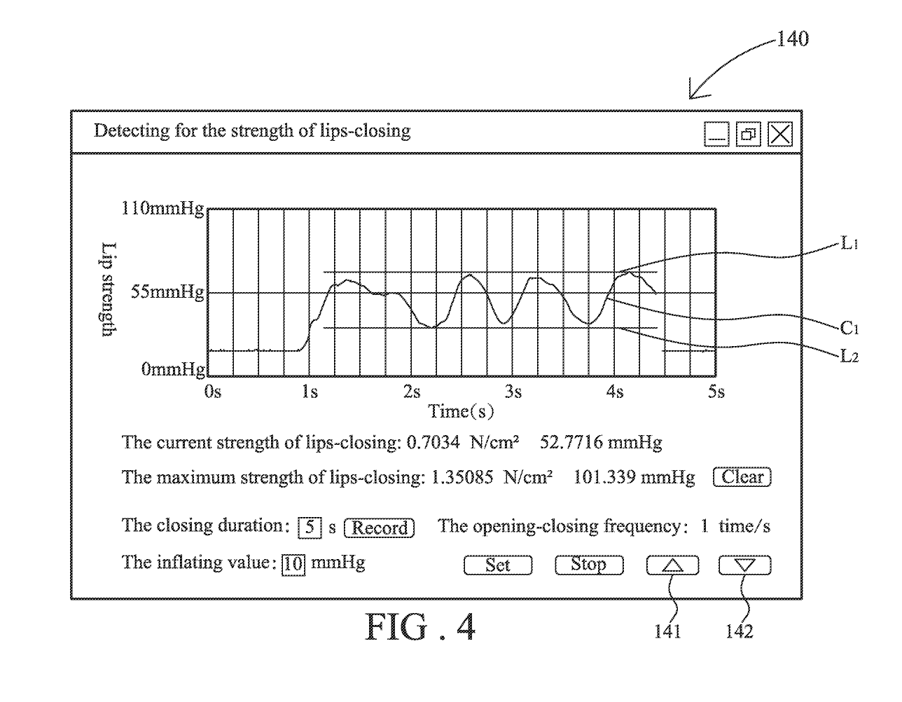

Finally, back to step S16, the user interface 140 shown as FIG. 4 displays the closing duration, the opening-closing frequency, the current strength of lips-closing and the maximum strength of lips-closing thereon, for medical staff further analysis and determining the treatment status of the TMD. For patient training of step S17, the user interface 140 of FIG. 4 provides two buttons 141, 142 for setting an upper bound and a lower bound of the real-time force curve C₁. In addition, the user interface 140 also shows some real-time patterns such as two lines L₁, L₂ for designating the upper bound and the lower bound respectively. During therapeutic training sessions, if the lips strength is higher than the upper bound, the user must open his/her lips; and if the lips strength is lower than the lower bound, the user must close his/her lips. Thereby, the user can control the strength of lips-closing to maintain the real-time force curve between the upper bound and the lower bound.

The foregoing description of the preferred embodiment of the invention has been presented for purposes of illustration and description. It is not intended to be exhaustive or to limit the invention to the precise form or to exemplary embodiments disclosed. Accordingly, the foregoing description should be regarded as illustrative rather than restrictive. Obviously, many modifications and variations will be apparent to practitioners' skills. The embodiments are chosen and described in order to best explain the principles of the invention and its best mode for practical application, thereby to help user to understand the invention formed by various embodiments and with various modifications as are suited to the particular use or implementation contemplated. It is intended that the scope of the invention be defined by the claims appended hereto and their equivalents in which all terms are meant in their broadest reasonable sense unless otherwise indicated. Therefore, the term “the invention”, “the present invention” or the like is not necessary limited the claim scope to a specific embodiment, and the reference to particularly preferred exemplary embodiments of the invention does not imply a limitation on the invention, and no such limitation is to be inferred. The invention is limited only by the spirit and scope of the appended claims. The abstract of the disclosure is provided to comply with the rules requiring an abstract, which will allow a searcher to quickly ascertain the subject matter of the technical disclosure of any patent issued from this disclosure. It is submitted with the understanding that it will not be used to interpret or limit the scope or meaning of the claims. Any advantages and benefits described may not apply to all embodiments of the invention. It should be appreciated that variations may be made in the embodiments described by persons' skills without departing from the scope of the present invention as defined by the following claims. Moreover, no element and component in the present disclosure is intended to be dedicated to the public regardless of whether the element or component is explicitly recited in the following claims.

What is claimed is:

1. An apparatus for detecting a strength of lips-closing comprising:
an air bag placed between the lips of an user, wherein the air bag can be inflated or deflated to change the pressure therein;
a pressure sensing unit connected to the air bag to detect a pressure exerted on the air bag by the user closing the lips, so as to produce a signal; and
a micro-controller module, electrical connected to the pressure sensing unit, for converting the signal into the strength of lips-closing.

2. The apparatus for detecting a strength of lips-closing of claim 1, further comprising a pump and a deflating valve, wherein the pump and the deflating valve are electrically connected to the micro-controller module, and the micro-controller module controls the pump to inflate the air bag or controls the deflating valve to deflate the air bag.

3. The apparatus for detecting a strength of lips-closing of claim 2, further comprising an user interface electrically connected to the micro-controller module for the user to operate the micro-controller module, thereby control the air bag inflated or deflated for adjusting the pressure therein to achieve a predetermined pressure value.

4. The apparatus for detecting a strength of lips-closing of claim 3, further comprising a record unit and a database, electrically connected to the user interface, the record unit records an closing duration as the lips of the user exerting the pressure on the air bag and an opening-closing frequency of the lips, and the record unit records the strength of lips-closing processed by the micro-controller module, thereby obtain a current strength of lips-closing and a maximum strength of lips-closing, and stores the closing duration, the opening-closing frequency, the current strength of lips-closing and the maximum strength of lips-closing in the database.

5. The apparatus for detecting a strength of lips-closing of claim 3, wherein the user interface has a real-time visual feedback mechanism.

6. The apparatus for detecting a strength of lips-closing of claim 1, further comprising an amplification unit electrically connected between the pressure sensing unit and the micro-controller module to amplify the signal produced by the pressure sensing unit.

7. The apparatus for detecting a strength of lips-closing of claim 1, further comprising a filter unit electrically connected between the pressure sensing unit and the micro-controller module to filter the signal produced by the pressure sensing unit.

8. The apparatus for detecting a strength of lips-closing of claim 1, wherein the micro-controller module comprises a record unit for recording an closing duration as the lips of the user exerting the pressure on the air bag and an opening-closing frequency of the lips, wherein the record unit records the strength of the user processed by the micro-controller module, thereby obtain a current strength of lips-closing and a maximum strength of lips-closing, and stores the closing duration, the opening-closing frequency, the current strength of lips-closing and the maximum strength of lips-closing.

9. A method of detecting a strength of lips-closing, comprising steps of:
providing the apparatus for detecting the strength of lips-closing of claim 3;
setting the predetermined pressure value of the air bag;
controlling the inflating or deflating procedure of the air bag according to the predetermined pressure value;
placing the air bag within the lips of the user when the pressure in the air bag achieves the predetermined pressure value;
sensing the pressure exerted on the air hag by the lips of the user to generate a signal; and
converting the signal into the strength of lips-closing.

10. The method of detecting a strength of lips-closing of claim 9, further comprising steps of:
amplifying the signal; and
filtering the amplified signal.

11. The method of detecting a strength of lips-closing of claim 9, further comprising steps of:
recording a closing duration as the lips of the user forcing on the air bag, and recording an opening-closing frequency of the lips by means of a record unit;
recording the strength of lips-closing processed by the micro-controller module, thereby obtain a current strength of lips-closing and a maximum strength of lips-closing; and
storing the closing duration, the opening-closing frequency, the current strength of lips-closing and the maximum strength of lips-closing in a database by means of the record unit.

12. The method of detecting a strength of lips-closing of claim 9, further comprising steps of:
forming a real-time force curie according to the strength of lips-closing; and
providing a real-time visual feedback mechanism according to the real-time force curve.

13. The method of detecting a strength of lips-closing of claim 13, wherein the real-time visual feedback mechanism comprises steps of:
displaying two real-time patterns on the user interface to designate an upper bound and a lower bound of the real-time force curve; and
displaying the relative position of the real-time force curve, the upper bound and the lower bound on the user interface as the lips of the user exerting the pressure on the air bag.
